(19) United States
(12) Patent Application Publication
(10) Pub. No.: US 2026/0210823 A1
(43) Pub. Date: Jul. 23, 2026

(54) Deterioration Acceleration Method and Deterioration Acceleration Device

(71) Applicant: NTT, Inc., Tokyo (JP)

(72) Inventors: Kaori Negishi, Musashino-shi, Tokyo (JP); Takashi Miwa, Musashino-shi, Tokyo (JP); Akira Sugiyama, Musashino-shi, Tokyo (JP); Norihiro Fujimoto, Musashino-shi, Tokyo (JP); Manami Torimoto, Musashino-shi, Tokyo (JP)

(21) Appl. No.: 19/139,085

(22) PCT Filed: Dec. 23, 2022
(86) PCT No.: PCT/JP2022/047621

§ 371 Date: Jun. 13, 2025

(51) Int. Cl. G01N 3/34; G01N 17/00; G01N 33/44

(52) U.S. Cl. CPC G01N 3/34; G01N 17/004; G01N 33/442; G01N 2203/0005; G01N 2203/0023; G01N 2203/0066; G01N 2203/0641

(57) ABSTRACT

In a degradation acceleration method for accelerating composite degradation of a polymer material, a first step of applying, by an additional device (light source), an environmental degradation factor including light to the polymer material, a second step of applying, by a bending mechanism, a repeated bending stress to the polymer material, and a third step of measuring, by a measurement device (control device and infrared spectroscopic analysis device), a degradation progress state of the polymer material on the basis of an infrared spectroscopic analysis result of the polymer material are performed.

### DESCRIPTION

### Technical Field

The present disclosure relates to a degradation acceleration method and a degradation acceleration apparatus.

### Background Art

Polymer materials easily deteriorate and have low durability as compared with inorganic compounds such as metal, ceramic, and glass. It is considered that degradation of a polymer material is affected by three types of conditions: “environmental conditions” such as ultraviolet rays, heat, water, and chemicals; “mechanical conditions” such as tension, compression, bending, and impact; and “special conditions” such as electrical action (Non Patent Literature 1).

Degradation of polymeric materials under those individual conditions has long been studied. For example, Non Patent Literature 2 discloses degradation behavior due to light and heat. Non Patent Literature 3 discloses degradation behavior due to infrared rays. Non Patent Literature 4 discloses degradation behavior due to bending stress.

### CITATION LIST

### Non Patent Literature

Non Patent Literature 1: Ohishi, “Degradation Analysis, Durability Evaluation and Life-estimation for Reliability of Polymeric Materials,” Polymer, Vol. 48, November issue, 1999, p. 838-p. 841
Non Patent Literature 2: Miwa et al., “Comparison of Photo Degradation Behavior of LDPE Using Accelerated Weathering Instruments,” Material and Environments, 64, 2015, p. 139-p. 144
Non Patent Literature 3:“Evaluating method of weathering conditions for plastics using Polyethylene Reference Specimens (revised on Apr. 1, 2009) ,” Japan Weathering Test Center, JWTCS4002: 2009
Non Patent Literature 4: Yokoi et al., “Improvement of Folding Endurance of Molded Hinge Parts by Actuating the Movable Pin in a Mold,” Production Research 393, Vol. 42, No. 6, June 1990, p. 97-p. 100
Non Patent Literature 5: “Environmental Stress Cracking of Plastics,” Osaka Research Institute of Industrial Science and Technology, Technical Sheet, NO. 98037

### SUMMARY OF INVENTION

### Technical Problem

On the other hand, degradation of a polymer material under a plurality of conditions has been less reported except for Non Patent Literature 5 regarding environmental stress cracking of plastic. In particular, a report of composite degradation of a polymer material due to application of an environmental degradation factor and a bending stress cannot be confirmed. At present, in the case of accelerating degradation occurring under such conditions together, an operation of replacing a polymer material in each weather resistance test apparatus prepared for each condition occurs, and thus there is a problem of inefficiency.

The present disclosure has been made in view of the above circumstances, and an object of the present disclosure is to provide a technique capable of efficiently accelerating composite degradation of a polymer material.

### Solution to Problem

A degradation acceleration method of an aspect of the present disclosure is a degradation acceleration method for accelerating composite degradation of a polymer material, the method including: a first step of applying, by an additional device, an environmental degradation factor including light to the polymer material; a second step of applying, by a bending mechanism, a repeated bending stress to the polymer material; and a third step of measuring, by a measurement device, a degradation progress state of the polymer material on the basis of an infrared spectroscopic analysis result of the polymer material.

A degradation acceleration apparatus of one aspect of the present disclosure is a degradation acceleration apparatus for accelerating composite degradation of a polymer material, the degradation acceleration apparatus including: an additional device configured to apply an environmental degradation factor including light to the polymer material; a bending mechanism configured to apply a repeated bending stress to the polymer material; and a measurement device configured to measure a degradation progress state of the polymer material on the basis of an infrared spectroscopic analysis result of the polymer material.

### Advantageous Effects of Invention

According to the present disclosure, it is possible to provide a technique capable of efficiently accelerating composite degradation of a polymer material.

### BRIEF DESCRIPTION OF DRAWINGS

FIG. 1 is a side view illustrating an overall configuration of a degradation acceleration apparatus.

FIG. 2 is a cross-sectional view along line A-A′ illustrated in FIG. 1.

FIG. 3 is a perspective view illustrating configurations of a support member, an upper fixture, a lower fixture, a test specimen, and a bending jig.

FIG. 4 is a perspective view illustrating a state in which the support member illustrated in FIG. 3 is bent.

FIG. 5 is a perspective view illustrating a state in which the bending jig illustrated in FIG. 3 is moved to a retracted position.

FIG. 6 is a block diagram illustrating configurations of a control device, an infrared spectroscopic analysis device, and each device connected to the control device and the infrared spectroscopic analysis device.

FIG. 7 is a flowchart illustrating an operation of the degradation acceleration apparatus.

FIG. 8 is an explanatory view illustrating a state in which light from a light source is radiated to a test specimen.

FIG. 9 is an explanatory view illustrating a state in which light from the light source is radiated to the test specimen.

FIG. 10 is an explanatory view illustrating a state in which light from the light source is radiated to the test specimen.

FIG. 11 is an explanatory view illustrating test results of degradation acceleration.

FIG. 12 is an explanatory view illustrating test results of degradation acceleration.

FIG. 13 is a block diagram illustrating a hardware configuration of a control device.

### DESCRIPTION OF EMBODIMENTS

Hereinafter, an embodiment will be described with reference to the drawings.

### Overview of Present Disclosure

The present disclosure discloses a method and apparatus for accelerating composite degradation of a polymeric material. Specifically, composite deterioration of a polymer material is accelerated by applying an environmental degradation factor including light imitating sunlight and a repeated bending stress to the polymer material. Composite degradation is accelerated, and a degradation progress state of the polymer material is measured by infrared spectroscopic analysis.

### Configuration of Degradation Acceleration Apparatus

A degradation acceleration apparatus according to the present embodiment is an apparatus for accelerating composite degradation of a polymer material. Polymer materials are plastic materials that have flexibility to the extent of being bendable and are assumed to be degraded by ultraviolet rays. Polymer materials include, for example, polyethylene and polypropylene.

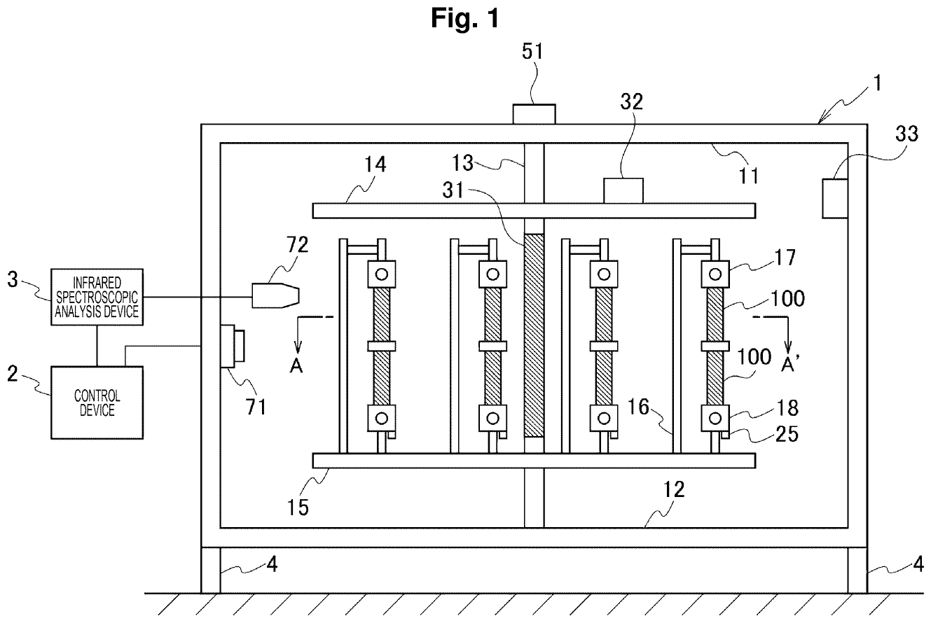

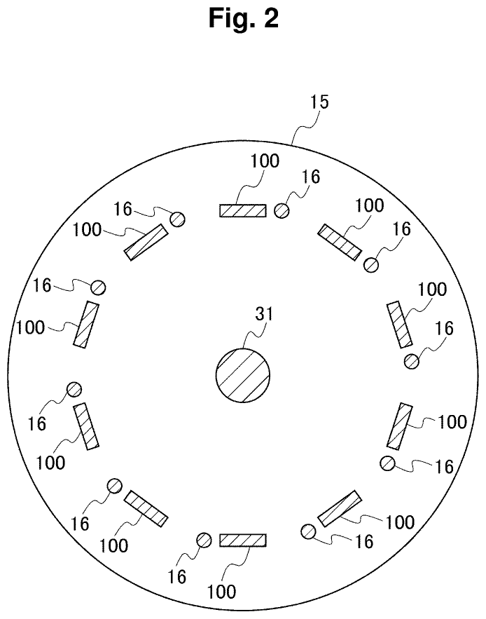

FIG. 1 is a side view illustrating an overall configuration of the degradation acceleration apparatus according to the present embodiment. FIG. 2 is a cross-sectional view along line A-A′ illustrated in FIG. 1. As illustrated in FIG. 1, the degradation acceleration apparatus includes a housing 1, a control device 2 disposed in the vicinity of a side portion of the housing 1, and an infrared spectroscopic analysis device 3 disposed in the vicinity of the control device 2.

### Configuration and Function of Housing 1

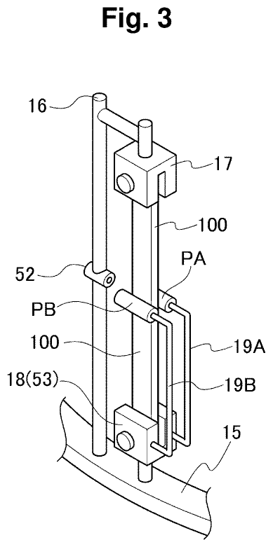

The housing 1 includes an additional device (a light source 31, a temperature regulator 32, and a humidity regulator 33) that applies environmental degradation factors including light imitating sunlight to a test specimen 100 of a polymer material, a mechanism part (a rotation mechanism 51, a bending mechanism 52, and a drive mechanism 53) that applies repeated bending stress and the like to the test specimen 100, and an observation part (an imaging unit 71, and a measurement unit 72) for observing a degradation progress state of the test specimen 100. The bending mechanism 52 and the drive mechanism 53 are illustrated in FIG. 3 to FIG. 5.

As illustrated FIG. 1, the housing 1 has a hollow rectangular shape and has a sealed internal structure. Legs 4 are provided at the lower portion of housing 1. The housing 1 is supported by the legs 4 and mounted on a flat floor surface.

A support 13 that connects the central portion of a top plate 11 and the central portion of a bottom plate 12 is installed Inside the housing 1. An upper disk 14 and a lower disk 15 (fixing members) are coupled to the support 13. A light source 31 is disposed between the upper disk 14 and the lower disk 15 in the support 13.

The light source 31 radiates light imitating sunlight to the surroundings (in 360° radially). That is, the light source 31 is installed at the central portion of the lower disk 15 (fixing member), and radiates light to a plurality of test specimens 100. The light source 31 radiates light including light in an ultraviolet region, for example, to the test specimens 100 present in the surroundings. The light source 31 may radiate light in a visible region or an infrared region.

The light source 31 is, for example, an ultraviolet fluorescent lamp, a xenon lamp, a sunshine carbon lamp, or a metal halide lamp. The light source 31 may be a combination of such lamps and a filter for bringing a spectroscopic radiation distribution of radiation light close to sunlight. As the light source 31, one that performs a test in accordance with “ISO 11341,” “JIS K 5600-7-7,” “JIS K 7350-2,” or the like can be used.

The temperature regulator 32 that maintains the inside of the housing 1 at a predetermined humidity is provided on the upper surface of the upper disk 14. The humidity regulator 33 that maintains the inside of the housing 1 at a predetermined temperature is provided on the inner side surface of the housing 1.

The support 13 is rotatable with the longitudinal direction as a rotation axis by a rotation mechanism 51. That is, by controlling the rotation mechanism 51, it is possible to cause the upper disk 14 and the lower disk 15 to rotate about the support 13 (central axis).

The rotation mechanism 51 is equipped with, for example, a stepping motor, and can rotate the upper disk 14 and the lower disk 15 and stop the same at a predetermined angle. The rotation mechanism 51 controls the plurality of test specimens 100 to be sequentially positioned at imaging ranges/measurement positions for imaging and measurement by the imaging unit 71 and the measurement unit 72 by rotating the lower disk 15 (fixing member).

As illustrated in FIG. 2, a plurality of (10 in this example) support members 16 are erected at equal intervals in the circumferential direction on the upper surface of the lower disk 15. A lower fixture 18 (refer to FIG. 1) is mounted in the vicinity of the side portion of each support member 16, and an upper fixture 17 (refer to FIG. 1) paired with the lower fixture 18 is mounted at a position above the lower fixture 18. The upper fixture 17 is coupled to and supported by an upper end portion of the support member 16.

FIG. 3 is a perspective view illustrating configurations of the support members 16, and the upper fixture 17, the lower fixture 18, and test specimens 100 mounted in the vicinity of the side portions of the support members 16. As illustrated in FIG. 3, the test specimen 100, which is strip-shaped, is disposed between the upper fixture 17 and the lower fixture 18. That is, each test specimen 100 is mounted on the circumference of the lower disk 15 (fixing member) having a circular shape (refer to FIG. 2).

The upper end portion of the test specimen 100 is coupled to the upper fixture 17. The lower end portion of the test specimen 100 is coupled to the lower fixture 18. That is, the test specimen 100 can be attached and detached by operating the upper fixture 17 and the lower fixture 18.

The bending mechanism 52 is mounted substantially at the center of the support member 16 in the vertical direction. The bending mechanism 52 bends the support member 16 at a predetermined angle under the control of the control device 2. For example, the bending mechanism 52 bends the support member 16 to ±30°, ±60°, ±90°, ±120°, or ±180°.

Two bending jigs 19A and 19B are coupled to the lower fixture 18. End portions PA and PB of the bending jigs 19A and 19B have a cylindrical shape. The end portions PA and PB of the two bending jigs 19A and 19B can clamp a substantially central portion of the test specimen 100.

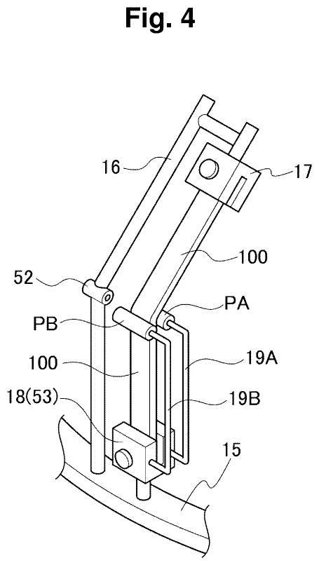

Therefore, when the support member 16 is bent by controlling the bending mechanism 52, the test specimen 100 can be bent. That is, as illustrated in FIG. 3, the central portion of the test specimen 100 is clamped by the end portions PA and PB of the two bending jigs 19A and 19B. Therefore, when the support member 16 is bent at a predetermined angle, the test specimen 100 coupled to the upper fixture 17 is bent at a predetermined angle with the portion clamped by the end portions PA and PB as a fulcrum as illustrated in FIG. 4. That is, the bending mechanism 52 switches extension and bending of the test specimen 100 and applies bending stress.

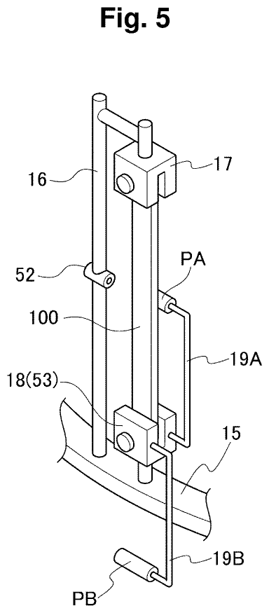

The lower fixture 18 is equipped with a drive mechanism 53. The drive mechanism 53 individually retracts the two bending jigs 19A and 19B under the control of the control device 2. Specifically, as illustrated in FIG. 5, the drive mechanism 53 can flip down and retract the bending jig 19B. Similarly with respect to the bending jig 19A, the drive mechanism 53 can flip down and retract the bending jig 19A.

Hereinafter, the positions where the bending jigs 19A and 19B are flipped downward are referred to as “retracted positions.” A position where the bending jigs 19A and 19B clamp the test specimen 21 is referred to as an “operation position.”

By moving the bending jig 19A on the inner side (the side of the light source 31) between the bending jigs 19A and 19B to the retracted position, blocking of light radiated from the light source 31 to the test specimen 100 by the bending jig 19A is prevented. By moving the bending jig 19B on the outer side to the retraction position, the bending jig 19B is prevented from becoming an obstacle when the imaging unit 71 and the measurement unit 72 image and measure the test specimen 100. That is, the drive mechanism 53 can drive (move) each of the bending jigs 19A and 19B to one of the operation position and the retracted position.

Referring back to FIG. 1, the imaging unit 71 is mounted at an appropriate portion on a side surface on the inside of the housing 1. That is, the imaging unit 71 is mounted on the periphery of the lower disk 15 (fixing member). The imaging unit 71 is, for example, an optical camera, a CCD camera, or a microscope. The imaging unit 71 is mounted at a position where the vicinity of the central portion of the test specimen 100 can be imaged. That is, the test specimen 100 is imaged by the imaging unit 71.

The measurement unit 72 is disposed inside the hollow space of the housing 1. That is, the measurement unit 72 is disposed around the lower disk 15 (fixing member). The measurement unit 72 is attached to the infrared spectroscopic analysis device 3 via an optical fiber. The measurement unit 72 is pressed against the test specimen 100 under the control of the infrared spectroscopic analysis device 3, and functions as a portion for acquiring light radiated to the test specimen 100.

### Configurations and Functions of Control Device 2 and Infrared Spectroscopic Analysis Device 3

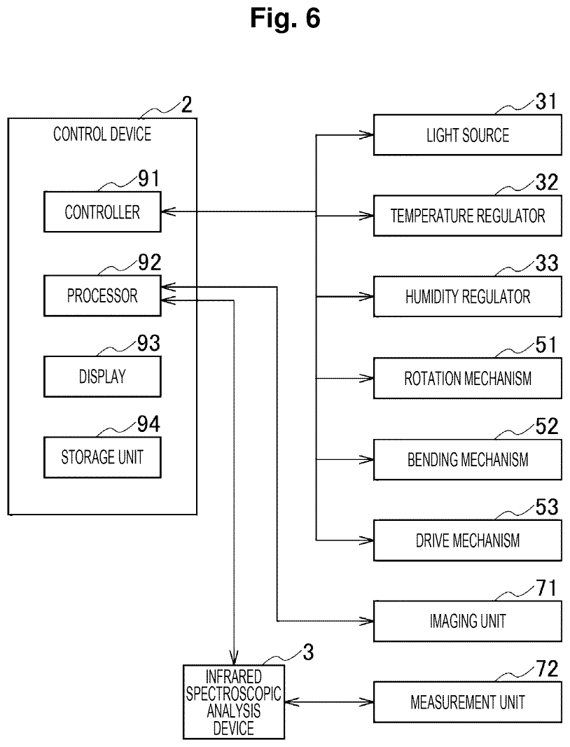

FIG. 6 is a block diagram illustrating configurations of the control device 2, the infrared spectroscopic analysis device 3, and devices connected to the control device 2 and the infrared spectroscopic analysis device 3. The devices are the above-described additional device (light source 31, temperature regulator 32, and humidity regulator 33), the mechanism part (rotation mechanism 51, bending mechanism 52, and drive mechanism 53), and observation part (Imaging unit 71 and measurement unit 72). The control device 2 and the infrared spectroscopic analysis device 3 may be physically configured as one measurement device.

The infrared spectroscopic analysis device 3 is connected to a measurement unit 72 via an optical fiber, and is also electrically and physically connected to the control device 2. The infrared spectroscopic analysis device 3 is, for example, a Fourier transform infrared spectrophotometer in accordance with “JIS K 0117.” The infrared spectroscopic analysis device 3 may be any analysis device capable of performing infrared spectroscopic analysis of the test specimen 100.

The infrared spectroscopic analysis device 3 is a device that performs infrared spectroscopic analysis of the test specimen 100. Only the optical fiber is introduced into the housing 1, and the infrared spectroscopic analysis device 3 presses the measurement unit 72 against the test specimen 100 at the time of measurement and measures the absorbance of each of the carbonyl group and the methylene group absorbed by the test specimen 100 via the optical fiber. The infrared spectroscopic analysis device 3 outputs a measured value of each absorbance to the control device 2. The carbonyl group and the methylene group are examples of observation data. Any data may be used as long as the data can measure a degradation progress state of the test specimen 100.

The control device 2 is electrically and physically connected to the light source 31, the temperature regulator 32, the humidity regulator 33, the rotation mechanism 51, the bending mechanism 52, the drive mechanism 53, and the imaging unit 71. As illustrated in FIG. 6, the control device 2 includes a controller 91, a processor 92, a display 93, and a storage unit 94.

The controller 91 controls on/off and illuminance of the light source 31.

The controller 91 controls the temperature regulator 32 such that the temperature in the housing 1 becomes a predetermined temperature.

The controller 91 controls the humidity regulator 33 such that the humidity in the housing 1 becomes a predetermined humidity.

The controller 91 controls the rotation mechanism 51 to rotate the support 13 and thus rotate the upper disk 14 and the lower disk 15. For example, the controller 91 controls ten test specimens 100 installed on the lower disk 15 to be located in the imaging range of the imaging unit 71 and the measurement position of the measurement unit 72 by rotating the support 13 at intervals of 36°.

The controller 91 controls the bending mechanism 52 provided for each of the ten test specimens 100 such that the support member 16 corresponding to each test specimen 100 is bent at a predetermined angle. For example, the controller 91 controls bending angles of the ten test specimens 100 to be 30°, 60°, 90°, 120°, 180°, −30°, −60°, −90°, −120°, and −180°. The bending mechanism 52 may be configured to be able to perform tests in accordance with “JIS P8115,” “JIS-K5600,” “JIS C 3005,” or the like.

The bending angle of each test specimen 100 can be arbitrarily set. For example, all the bending angles of the ten test specimens 100 may be set to the same angle, or the ten test specimens 100 may be divided into groups of five test specimens, and the same angle may be set for the test specimens 100 in each group. That is, the plurality of test specimens 100 may be divided into test specimens to be bent at a first angle and test specimens to be bent at a second angle, and the bending mechanism 52 may extend and bend each test specimen 100 at the angle corresponding thereto. Alternatively, different bending angles may be set for all the ten test specimens 100.

The controller 91 controls the bending mechanism 52 provided for each of the ten test specimens 100 such that the number of times of bending in which the support member 16 corresponding to each test specimen 100 repeats bending and stretching becomes a predetermined number. For example, the controller 91 controls the number of times of bending and stretching of the ten test specimens 100 to be 5 times, 10 times, 15 times, and 20 times.

The number of times of bending and stretching of each test specimen 100 can be arbitrarily set. For example, the number of times of bending and stretching of all the ten test specimens 100 may be set to be the same, or the ten test specimens 100 may be divided into groups of five test specimens and the numbers of times of bending and stretching of the groups may be set to be the same. That is, the plurality of test specimens 100 may be grouped into test specimens 100 for which stretching and bending is repeated the first number of times and test specimens 100 for which stretching and bending is repeated the second number of times, and the bending mechanisms 52 may stretch and bend each test specimen 100 the number of time of bending and stretching set for each group. Alternatively, different numbers of times of bending and stretching may be set for the ten test specimens 100.

The controller 91 controls the drive mechanism 53 such that the two bending jigs 19A and 19B are at either the operation position or the retraction position described above.

The processor 92 images the test specimen 100 by controlling the imaging unit 71. The processor 92 acquires a captured image of the test specimen 100.

The processor 92 acquires a measured value of infrared spectroscopic analysis of the test specimen 100 from the infrared spectroscopic analysis device 3. That is, the processor 92 acquires measured values of absorbances of the carbonyl group and the methylene group absorbed in the test specimen 100. Then, the processor 92 calculates the carbonyl index value (=the ratio of the absorbance of the carbonyl group to the absorbance of the methylene group) of the test specimen 100 based on the acquired absorbances of the carbonyl group and the methylene group of the test specimen 100, and measures a degradation progress state of the test specimen 100 based on the calculated carbonyl index value and the captured image (image information). For example, the processor 92 acquires an image of the test specimen 100 captured by the imaging unit 71, and measures the depth of a crack generated in the test specimen 100 by analyzing the image.

The processor 92 determines whether or not the carbonyl index value of the test specimen 100 has increased to a threshold value or more, determines whether or not the depth of the crack of the test specimen 100 has progressed to a threshold value or more, and controls the presence or absence of application of an environmental degradation factor by the additional device (light source 31, temperature regulator 32, and humidity regulator 33) and the presence or absence of application of a bending stress or the like by the mechanism part (rotation mechanism 51, bending mechanism 52, and drive mechanism 53) according to each determination result. For example, the processor 92 switches lighting of the light source 31 to turn-off. For example, the processor 92 stops application of bending stress by the bending mechanism 52.

That is, when it is determined that the carbonyl index value of the test specimen 100 or the depth of the crack of the test specimen 100 is equal to or greater than the preset value, or when the total weather resistance test time is equal to or greater than a preset time, the processor 92 stops the degradation acceleration apparatus, and otherwise, progresses the degradation acceleration test.

The display 93 displays, for example, a captured image of the test specimen 100, and various measured values such as the thickness of the test specimen 100 and the depth of a crack. The display 93 may display information indicating an alarm when the depth of the crack becomes a predetermined value or more.

The storage unit 94 stores, for example, a captured image of the test specimen 100 and various measured values such as the thickness of the test specimen 100 and the depth of a crack.

### Operation of Degradation Acceleration Apparatus

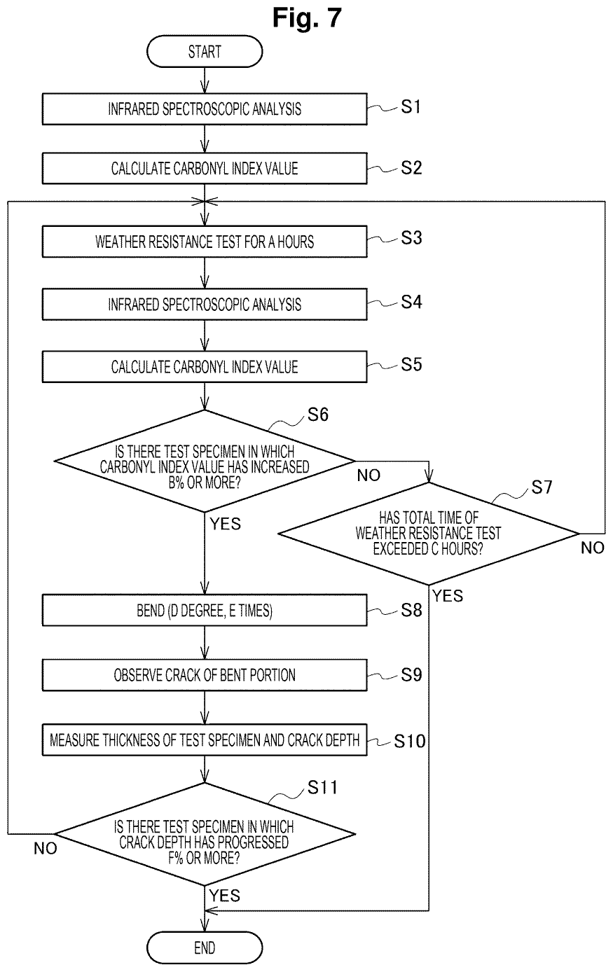

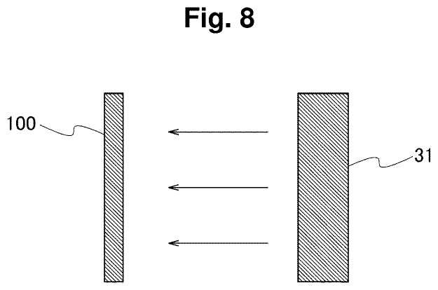

Next, the operation of the degradation acceleration apparatus according to the present embodiment configured as described above will be described with reference to the flowchart illustrated in FIG. 7 and operation diagrams illustrated in FIG. 8 to FIG. 10. Note that weather resistance test conditions and bending conditions can be designated according to the purpose, and various patterns are conceivable, and thus various setting values and the like are not limited to the following.

### Step S1;

First, the infrared spectroscopic analysis device 3 performs infrared spectroscopic analysis of test specimens 100. Specifically, the control device 2 controls the rotation mechanism 51 to move any one of ten test specimens 100 to the front of the measurement unit 72. The infrared spectroscopic analysis device 3 presses the measurement unit 72 against the test specimen 100, and measures the absorbance of each of the carbonyl group and the methylene group absorbed by the test specimen 100. The infrared spectroscopic analysis device 3 also measures each absorbance with respect to the remaining test specimens 100.

### Step S2;

Next, the control device 2 calculates an initial value of a carbonyl index value of each test specimen 100 using Formula (1) on the basis of the measured absorbances of the carbonyl group and the methylene group of each test specimen 100.

$$CI = A / B \qquad ( 1 )$$

CI is a carbonyl index value. A is the absorbance (=I−I₀) of the carbonyl group around 1715 cm¹. B is the absorbance (=I−I₀) of the methylene group around 2020 cm¹. I is the absorbance at each wavenumber. I₀ is the baseline absorbance by the baseline method for each wavenumber. The calculation formula for the carbonyl index value is not limited to Formula (1) since the absorption position may be shifted back and forth depending on the state of the test specimen 100 and measurement conditions.

### Step S3;

Next, the control device 2 controls the light source 31, the temperature regulator 32, and the humidity regulator 33 to perform a weather resistance test for A hours under preset conditions. For example, the weather resistance test is set in accordance with “JIS K 5600-7-7” and “JIS K 7350-2”′ at a black panel temperature of 63° C., an inside temperature of 38° C., and a humidity of 50% RH. The water spray time is 18 minutes in 2 hours. For example, as illustrated in FIG. 8, the light source 31 is turned on to continuously radiate light imitating sunlight to the entire periphery (360° direction) around the light source 31 for a certain period of time. The ultraviolet radiation intensity at a wavelength of 300 nm to 400 nm is 60 W/m². Under such conditions, accelerated degradation of the test specimen 100 is performed for 100 hours.

### Step S4;

Next, the infrared spectroscopic analysis device 3 performs infrared spectroscopic analysis of the test specimen 100 again. Specifically, the infrared spectroscopic analysis device 3 re-measures the absorbance of each of the carbonyl group and the methylene group of each test specimen 100.

### Step S5;

Next, the control device 2 calculates a carbonyl index value of each test specimen 100 after the weather resistance test is performed for A hours based on the remeasured absorbances of the carbonyl group and the methylene group.

### Step S6;

Next, the control device 2 calculates, for each test specimen 100, how many percent the carbonyl index value after the weather resistance test is increased with respect to the carbonyl index value before the weather resistance test. The control device 2 determines whether or not there is a test specimen 100 in which the carbonyl index value is increased by B % (for example, 0.1%) or more set in advance by a user.

### Step S7;

When there is no test specimen 100 in which the carbonyl index value has increased by B % or more, the control device 2 determines whether or not the total time of the weather resistance test has exceeded a designated C hours (for example, 3000 hours). When the total time of the weather resistance test does not exceed C hours, the process returns to step S3. When the total time of the weather resistance test exceeds C hours, all processes are terminated.

### Step S8;

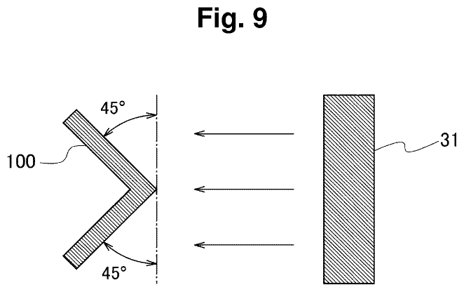

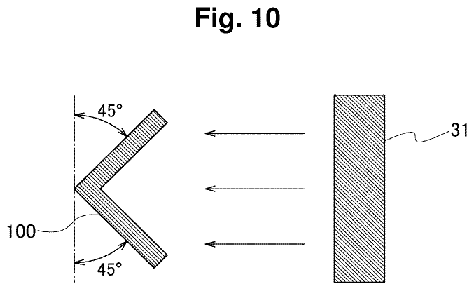

When there is a test specimen 100 in which the carbonyl index value has increased by B % or more, the control device 2 controls the bending mechanism 52 to bend the test specimen 100 under preset conditions. For example, as illustrated in FIG. 9 and FIG. 10, the control device 2 controls the test specimen 100 to be D° (for example, ±30°, ±60°, ±90°, ±120°, or ±180°). The control device 2 repeats the bending and stretching operations a predetermined number E of times (for example, 10 times or 30 times).

### Step S9;

Next, the control device 2 images each test specimen 100 by controlling the imaging unit 71. For example, the control device 2 images a bent portion of each test specimen 100. The user observes the degradation state of the test specimen 100 from the captured image of each test specimen 100.

### Step S10;

Next, the control device 2 analyzes the captured image of each test specimen 100, and measures the thickness of each test specimen 100 and the depth of a crack generated in each test specimen 100.

### Step S11;

Finally, the control device 2 calculates, for each test specimen 100, how many percent the depth of a crack has progressed with respect to the test specimen 100 on the basis of the thickness of the test specimen 100 and the depth of the crack. The control device 2 determines whether or not there is a test specimen 100 having a calculated value greater than F % (for example, 50%) preset by the user. Then, in a case where there is no test specimen 100 having a calculated value greater than F %, the process returns to step S3. In a case where there is a test specimen 100 having a calculated value greater than F %, all the processes are terminated.

That is, the degradation acceleration apparatus repeats the loop of steps S3 to S11, for example, repeats bending of the test specimen 100 every time the carbonyl index value increases by 0.1%, and is stopped according to preset program branching conditions when a crack depths have become 50% or more with respect to the test specimen 100 in all test specimens 100 or when the total time of the weather resistance test exceeds 3000 hours, that is, when the number of loops of steps S3 to S11 reaches 30 times.

### Test Results of Degradation Acceleration

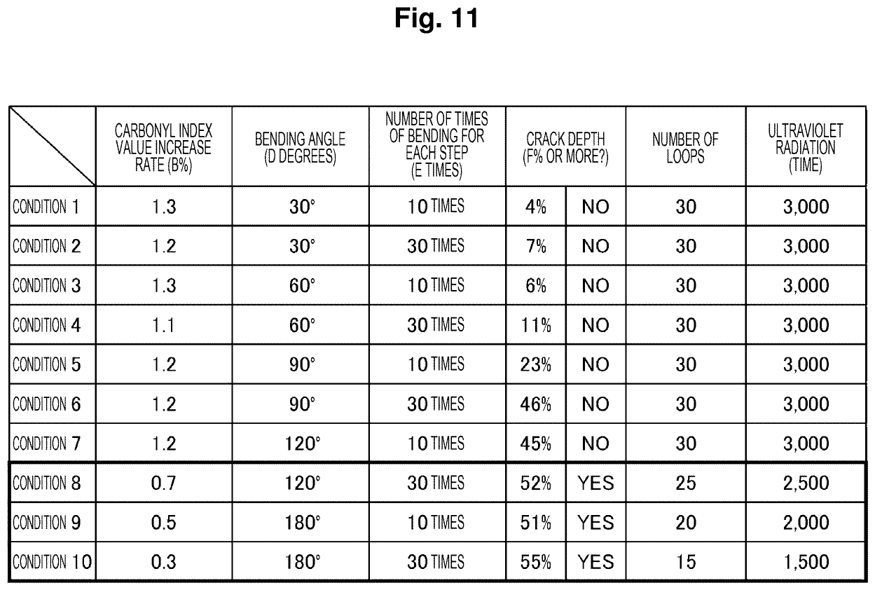

FIG. 11 shows examples of results of a plurality of conditions set for one type of test specimen, and a carbonyl index value increase rate and a crack depth when the degradation acceleration apparatus is stopped. As the test specimen, polypropylene having a size of about 4 cm×10 cm, a total thickness of about 5 mm, and a hinge portion thickness of about 1 mm was used. The outside of a bent portion of the test specimen was set so as to be irradiated with light, a bending angle was set to 30°, 60°, 90°, 120°, and 180°, and the number of times of bending was set to 10 and 30 times.

The test specimens under conditions 8 to 10 having large bending angles were determined to have a set crack depth of 50% or more at the stages of the number of loops of 25, 20, and 15, respectively. On the other hand, in the test specimens under conditions 1 to 7 having small bending angles, the ultraviolet radiation time is longer than that under conditions 8 to 10, but the crack depth is still less than the set value even after the carbonyl index value is increased through the number of loops of 30 times. From these test results, it is possible to efficiently separate the environmental degradation factor and the range in which the test specimens can withstand bending.

For example, from the above-described test results, it can be ascertained that the polymer material can be used outdoors for a long period of time if it is used at a bending angle of 90° or less. This cannot be easily inferred from a simple combination of the weather resistance test and the bending resistance test. Note that the bending angle, the number of times, and the like may be arbitrarily set according to the purpose, and are not limited to these numerical values.

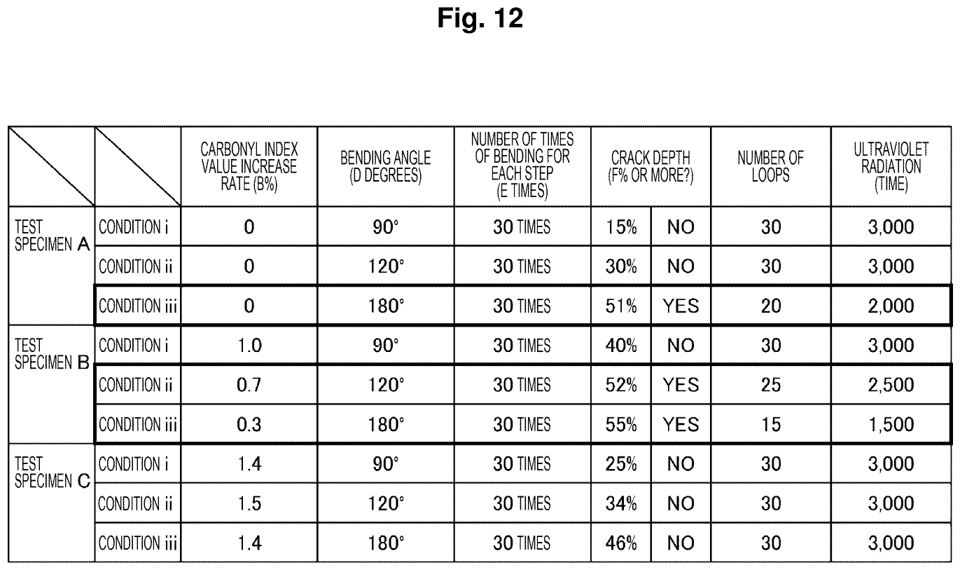

FIG. 12 shows examples of results of conditions set for three types of test specimens, and a carbonyl index value increase rate and a crack depth when the degradation acceleration apparatus is stopped. As the test specimen, polypropylene having a size of about 4 cm×10 cm, a total thickness of about 5 mm, and a hinge portion thickness of about 1 mm was used. The outside of bent portions of the test specimens was set so as to be irradiated with light, bending angles were set to 90°, 120°, and 180°, and the number of times of bending was set to 30 times.

It is ascertained that the crack depth of the test specimen A can be curbed to less than 50% in the case of bending up to 120 degrees. However, a test specimen in which the carbonyl index value does not increase in the first place like the test specimen A can be evaluated by the bending test alone without applying the degradation acceleration method of the present embodiment.

On the other hand, although the carbonyl index value of the test specimen B increased, it is ascertained that the crack depth was curbed to less than 50% when the test specimen B was bent up to 90 degrees. In addition, even when the test specimen C was bent up to 180 degrees, the crack depth was curbed to less than 50%. In any case, by setting an appropriate bending angle on the basis of evaluation results by the degradation acceleration method of the present embodiment, it is possible to use for a long period of time.

### Effects of Embodiment

According to the present embodiment, since the degradation acceleration apparatus includes the additional device (light source 31, temperature regulator 32, and humidity regulator 33) that applies an environmental degradation factor including light to the test specimen 100 of a polymer material, the mechanism part (rotation mechanism 51, bending mechanism 52, and drive mechanism 53) that applies repeated bending stress and the like to the test specimen 100, and the measurement device (control device 2 and infrared spectroscopic analysis device 3) that measures the degradation progression state of the test specimen 100 on the basis of infrared spectroscopic analysis results of the test specimen 100, the resistance of the polymer material to composite degradation due to light and bending stress can be easily and efficiently evaluated. £ In particular, it is evaluated that a degradation progress rate of varies depending on the bending angle, and the polymer material is used in the range of bending angles at which the progress of degradation is small, whereby the polymer material can be stably used for a long period of time.

### Others

The present disclosure is not limited to the embodiments described above, and it is obvious that many modifications and combinations can be implemented by a person having ordinary knowledge in the art within the technical idea of the present disclosure.

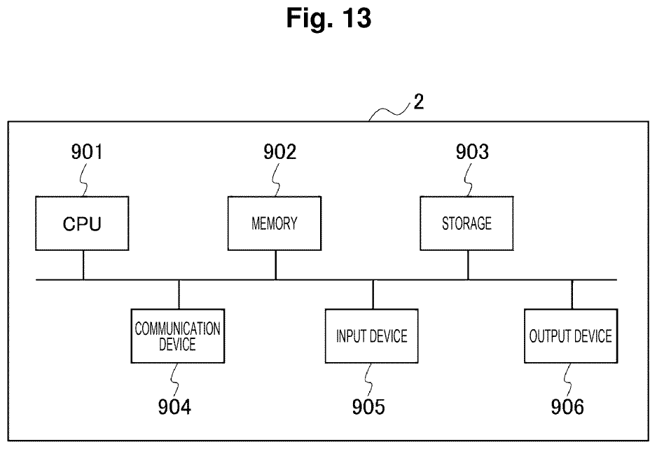

For example, as illustrated in FIG. 13, the control device 2 of the present embodiment described above can be realized using a general-purpose computer system including a CPU 901, a memory 902, a storage 903, a communication device 904, an input device 905, and an output device 906. The memory 902 and the storage 903 are storage devices. In the computer system, the CPU 901 executes a predetermined program loaded on the memory 902 to implement each function of the control device 2.

The control device 2 may be implemented by one computer. The control device 2 may be implemented by a plurality of computers. The control device 2 may be a virtual machine implemented on a computer. The program for the control device 2 can be stored in a computer-readable recording medium such as an HDD, an SSD, a USB memory, a CD, or a DVD. The computer-readable recording medium is, for example, a non-transitory recording medium. The program for the control device 2 can also be distributed via a communication network.

### REFERENCE SIGNS LIST

1 Housing
2 Control device
3 Infrared spectroscopic analysis device
4 Leg portion
11 Top plate
12 Bottom plate
13 Support
14 Upper disk
15 Lower disk
16 Support member
17 Upper fixture
18 Lower fixture
19A, 19B Bending jig
31 Light source
32 Temperature regulator
33 Humidity regulator
51 Rotation mechanism
52 Bending mechanism
53 Drive mechanism
71 Imaging unit
72 Measurement unit
91 Controller
92 Processor
93 Display
94 Storage unit
100 Test specimen
901 CPU
902 Memory
903 Storage
904 Communication device
905 Input device
906 Output device

1. A degradation acceleration method for accelerating composite degradation of a polymer material, the degradation acceleration method comprising:
a first step of applying, by an additional device, an environmental degradation factor including light to the polymer material;
a second step of applying, by a bending mechanism, a repeated bending stress to the polymer material; and
a third step of measuring, by a measurement device, a degradation progress state of the polymer material on the basis of an infrared spectroscopic analysis result of the polymer material.

2. The degradation acceleration method according to claim 1, wherein
a degradation progress state of the polymer material is measured on the basis of image information of the polymer material in the third step.

3. The degradation acceleration method according to claim 1, wherein
the infrared spectroscopic analysis result of the polymer material is
a carbonyl index value of the polymer material.

4. A degradation acceleration apparatus for accelerating composite degradation of a polymer material, the degradation acceleration apparatus comprising:
an additional device configured to apply an environmental degradation factor including light to the polymer material;
a bending mechanism configured to apply a repeated bending stress to the polymer material; and
a measurement device configured to measure a degradation progress state of the polymer material on the basis of an infrared spectroscopic analysis result of the polymer material.

5. The degradation acceleration apparatus according to claim 4, wherein the infrared spectroscopic analysis result of the polymer material is a carbonyl index value of the polymer material, and
the measurement device
determines whether the carbonyl index value of the polymer material has increased to a threshold value or more, determines whether a depth of a crack in the polymer material has progressed to a threshold value or more, and controls whether or not the environmental degradation factor is applied by the additional device and whether or not the bending stress is applied by the bending mechanism depending on each determination result.
